(19) United States
(12) Patent Application Publication
(10) Pub. No.: US 2016/0007845 A1
(43) Pub. Date: Jan. 14, 2016

(54) FUNDUS IMAGING APPARATUS, ABERRATION CORRECTION METHOD, AND STORAGE MEDIUM

(71) Applicant: CANON KABUSHIKI KAISHA, Tokyo (JP)

(72) Inventor: Tsutomu Utagawa, Yokohama-shi (JP)

(21) Appl. No.: 14/792,411

(22) Filed: Jul. 6, 2015

(30) Foreign Application Priority Data
Jul. 10, 2014 (JP) 2014-142431

(51) Int. Cl. A61B 3/00; A61B 3/12; A61B 3/10; A61B 3/14

(52) U.S. Cl. CPC A61B 3/0025; A61B 3/14; A61B 3/1225; A61B 3/102; A61B 3/1015

(57) ABSTRACT

A fundus imaging apparatus includes an imaging unit that captures images of a plurality of regions of a fundus of a subject's eye, an initial value determination unit that determines an initial value of an aberration correction value of a target region based on an aberration correction value of at least one calculated region having a calculated aberration correction value from among the plurality of regions, a measurement unit that measures an aberration of the target region based on the initial value, a calculation unit that calculates an aberration correction value of the target region based on a measurement result of the measurement unit, and an aberration correction unit that corrects the aberration of the target region using the aberration correction value of the target region.

### BACKGROUND

1. Field

Aspects of the present invention generally relate to a fundus imaging apparatus, an aberration correction method, and a storage medium.

2. Description of the Related Art

Imaging apparatuses using scanning laser ophthalmoscope (SLO) and low-coherence light interference have recently been developed as ophthalmological imaging apparatuses. Each of these imaging apparatuses two-dimensionally irradiates a fundus with laser light, and receives reflected light from the fundus, thereby capturing an image of the fundus.

The imaging apparatus using the low-coherence light interference is called an optical coherence tomography apparatus or an optical coherence tomography (OCT). In particular, such an imaging apparatus is used to obtain a tomographic image of a fundus or near the fundus. Various types of OCTs including a time domain OCT (TD-OCT) and a spectral domain OCT (SD-OCT) have been developed. In recent years, such an ophthalmological imaging apparatus has been further developed to achieve higher resolution as the numeric aperture (NA) of laser irradiation becomes higher.

However, when the ophthalmological imaging apparatus captures an image of a fundus, the image needs to be captured via an optical structure such as a cornea and a crystalline lens of the eye. With the higher resolution, image quality of the captured image is markedly affected due to aberration of the cornea and the crystalline lens.

In view of the foregoing, study of an optical system with an adaptive optics (AO) function that measures and corrects an eye aberration is in progress. More specifically, the optical systems including AO-SLO and AO-OCT have been studied. An example of the AO-OCT is discussed in Optics Express, by Y. Zhang, et al. Vol. 14, No. 10, 15 May 2006. Generally, such AO-SLO and AO-OCT measure a wavefront of an eye by using Shack-Hartmann wavefront sensor system. According to the Shack-Hartmann wavefront sensor system, measuring light is emitted onto the eye, and reflected light from the eye is received by a charge-coupled device (CCD) camera via a microlens array, thereby measuring the wavefront. A deformable mirror and a special phase modulator are driven such that the measured wavefront is corrected, and then an image of the fundus is captured using the deformable mirror and the special phase modulator. This enables the AO-SLO and the AO-OCT to capture high-resolution images.

Generally, the AO used in an ophthalmologic apparatus models an aberration measured by a wavefront sensor into a function such as Zernike function, and calculates a correction amount of a wavefront correcting device by using the function. There are cases where a complex shape needs to be corrected. In such a case, an aberration is modeled into a function having many orders to calculate a correction amount, and then the wavefront correcting device is controlled.

However, the calculation of the correction amount involves a substantially high processing load, causing a big problem of longer calculation time. To solve such a problem, Japanese Patent Application Laid-Open No. 2012-235834 discusses a technique. According to the technique, when an affected area is periodically observed for disease follow-up, a correction value used in the past imaging operation is used.

Moreover, Japanese Patent Application Laid-Open No. 2012-213513 discusses a technique for capturing a plurality of regions of a fundus of a subject's eye in order, with a view to obtaining images needed for diagnosis without excess or deficiency.

### SUMMARY

According to an aspect of the present invention, a fundus imaging apparatus includes an imaging unit configured to capture images of a plurality of regions of a fundus of a subject's eye, an initial value determination unit configured to determine an initial value of an aberration correction value of a target region based on an aberration correction value of at least one calculated region having a calculated aberration correction value from among the plurality of regions, a measurement unit configured to measure an aberration of the target region based on the initial value, a calculation unit configured to calculate an aberration correction value of the target region based on a measurement result of the measurement unit, and an aberration correction unit configured to correct the aberration of the target region using the aberration correction value of the target region.

Further features of the present disclosure will become apparent from the following description of exemplary embodiments with reference to the attached drawings.

### BRIEF DESCRIPTION OF THE DRAWINGS

FIG. 1 is a diagram illustrating a fundus imaging apparatus according to a first exemplary embodiment.

FIG. 2 is a schematic diagram illustrating a reflection-type liquid crystal optical modulator.

FIG. 3 is a sectional view illustrating a wavefront correcting device.

FIG. 4 is a diagram illustrating a state in which an image of a fundus is captured.

FIGS. 5A and 5B are enlarged views illustrating a liquid crystal display.

FIGS. 6A and 6B are diagrams illustrating a galvanometer scanner.

FIGS. 7A and 7B are schematic diagrams illustrating a wavefront sensor.

FIG. 8 is a diagram illustrating a charge-coupled device (CCD) sensor.

FIGS. 9A and 9B are schematic diagrams illustrating a measurement result of a wavefront having a spherical aberration.

FIG. 10 is a diagram illustrating a control unit.

FIG. 11 is a flowchart illustrating imaging processing.

FIG. 12 is a diagram illustrating an imaging region of a subject's eye.

FIG. 13 is a flowchart illustrating aberration correction processing in detail.

FIG. 14 is a diagram illustrating a relationship between the number of aberration corrections and an aberration amount.

FIG. 15 is a flowchart illustrating imaging processing according to a second exemplary embodiment.

FIGS. 16A and 16B are diagrams illustrating imaging regions.

FIGS. 17A, 17B, and 17C are diagrams each illustrating an imaging region for each imaging mode.

### DESCRIPTION OF THE EMBODIMENTS

When an ophthalmological imaging apparatus using an AO captures images of a plurality of regions of a fundus of a subject's eye in order, imaging processing needs to be performed a plurality of times. In this case, if the regions to be imaged are different from one another, aberrations differ. Thus, the imaging apparatus needs to correct the aberration each time an image is captured. Thus, time needed for aberration correction is required to be shortened as much as possible to enhance efficiency of ophthalmological treatment. The present exemplary embodiment aims to shorten time needed for aberration correction of an eye in a fundus imaging apparatus using an AO. Hereinafter, exemplary embodiments will be described, and these exemplary embodiments are not seen to be limiting.

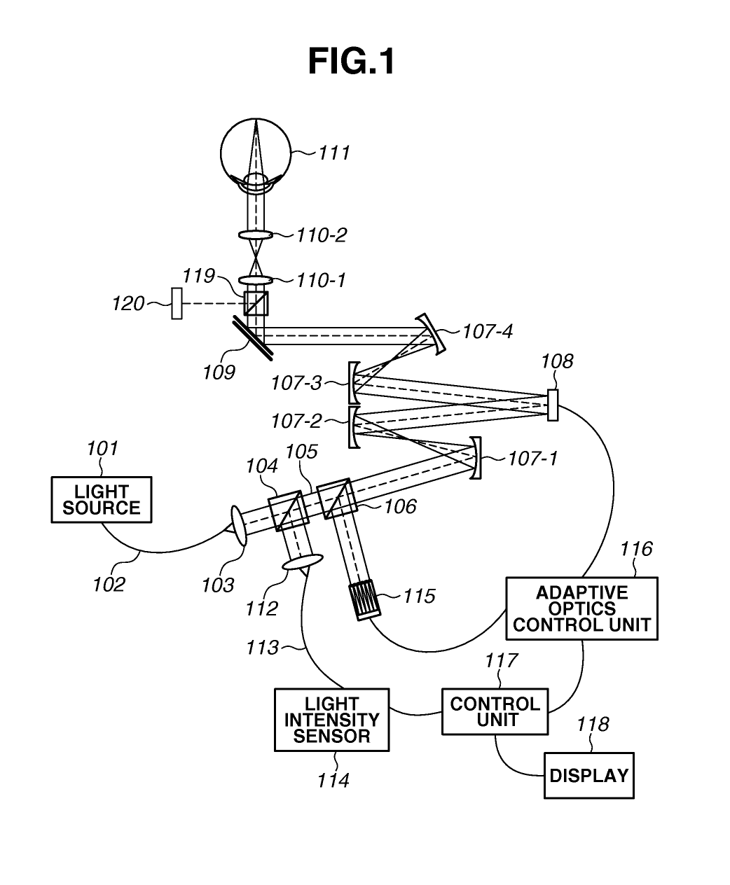

FIG. 1 is a diagram illustrating a fundus imaging apparatus according to a first exemplary embodiment. The fundus imaging apparatus of the present exemplary embodiment regards an eye as an examination target to be examined. The fundus imaging apparatus corrects an aberration occurring in the eye by using an adaptive optics system, and captures an image of a fundus.

In FIG. 1, a light source 101 is a super luminescent diode (SLD) light source having a wavelength of 840 nm. The wavelength of the light source 101 is not especially limited. However, the light source 101 for fundus imaging suitably has a wavelength of approximately 800 nm to 1500 nm to reduce glare of the light on the subject and to maintain resolution. In the present exemplary embodiment, the SLD light source is used. Alternatively, other light sources such as laser may be used. In the present exemplary embodiment, one light source is used to capture a fundus image and measure a wavefront. Alternatively, one light source for fundus imaging and another light source for wavefront measurement may be used. In such a case, lights are combined in a middle portion of an optical path.

The light emitted from the light source 101 passes a single-mode optical fiber 102, and is emitted as a parallel ray (measurement light 105) by a collimator 103. Then, the emitted measurement light 105 is transmitted through a light splitting unit 104 including a beam splitter, and guided to an adaptive optics system.

The adaptive optics system includes a light splitting unit 106, a wavefront sensor 115, a wavefront correcting device 108, and reflection mirrors 107-1 through 107-4. The reflection mirrors 107-1 through 107-4 guide light to the light splitting unit 106, the wavefront sensor 115, and the wavefront correcting device 108. The reflection mirrors 107-1 through 107-4 are arranged such that at least a pupil of an eye 111, the wavefront sensor 115, and the wavefront correcting device 108 have an optically conjugate relationship. In the present exemplary embodiment, a beam splitter is used as the light splitting unit 106.

After being transmitted through the light splitting unit 106, the measurement light 105 is reflected from the reflection mirrors 107-1 and 107-2 to enter the wavefront correcting device 108. The measurement light 105 reflected from the wavefront correcting device 108 is emitted onto the reflection mirror 107-3.

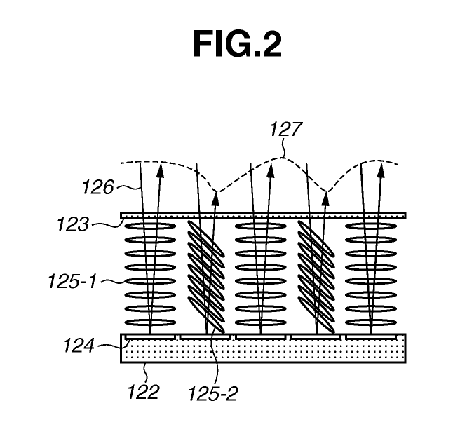

In the present exemplary embodiment, a spatial phase modulator with a liquid crystal device is used as the wavefront correcting device 108. FIG. 2 is a schematic diagram illustrating a reflection-type liquid crystal optical modulator. This modulator includes liquid crystal molecules 125 enclosed in a space between a base 122 and a cover 123. The base 122 includes a plurality of pixel electrodes, whereas the cover 123 includes transparent counter electrodes (not illustrated). When voltage is not applied to between the electrodes, the liquid crystal molecules 125 are oriented as liquid crystal molecules 125-1 illustrated in FIG. 2. When voltage is applied, the orientation of the liquid crystal molecules 125 changes. Hence, the liquid crystal molecules 125 are oriented as liquid crystal molecules 125-2 illustrated in FIG. 2. This changes a refractive index with respect to incident light.

Accordingly, voltage of each pixel electrode is controlled to change a refractive index of each pixel, so that a phase can be spatially modulated. For example, in a case where incident light 126 enters the reflection-type liquid crystal optical modulator, a phase of light passing through the liquid crystal molecules 125-2 lags behind that of light passing through the liquid crystal molecules 125-1. As a result, a wavefront 127 as illustrated in FIG. 2 is formed.

Generally, the reflection-type liquid crystal optical modulator includes several tens of thousands to several hundreds of thousands of pixels. Moreover, the liquid crystal optical modulator has polarization characteristics. Thus, the liquid crystal optical modulator may include a polarizing element for adjusting polarization of incident light.

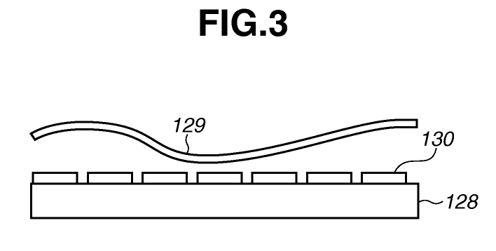

Alternatively, the wavefront correcting device 108 may be a deformable mirror that can locally change a reflecting direction of light. Various types of deformable mirrors are practically used. As one example of the deformable mirrors, FIG. 3 illustrates a sectional view of another wavefront correcting device 108. The wavefront correcting device 108 includes a deformable film-shaped mirror surface 129, a base 128, an actuator 130, and a support unit (not illustrated). The mirror surface 129 reflects incident light. The actuator 130 is arranged between the mirror surface 129 and the base 128. The support unit supports the mirror surface 129 from surroundings thereof.

The operating principles of the actuator 130 include the use of an electrostatic force, a magnetic force, and a piezoelectric effect. A configuration of the actuator 130 varies depending on the operating principles. On the base 128, a plurality of actuators 130 is two-dimensionally arrayed. The plurality of actuators 130 is selectively driven, so that the mirror surface 129 can be deformed. In general, a deformable mirror includes several tens to several hundreds of actuators.

In FIG. 1, the light reflected from the reflection mirrors 107-3 and 107-4 is one-dimensionally or two-dimensionally scanned by a scanning optical system 109. In the present exemplary embodiment, two galvanometer scanners for main scanning (a direction horizontal to the fundus) and sub-scanning (a direction vertical to the fundus) are used as the scanning optical system 109. Alternatively, in some cases, a resonant scanner is used for main scanning performed by the scanning optical system 109 so that an image is captured at higher speed. The fundus imaging apparatus may include an optical element such as a mirror and a lens between the scanners to cause each of the scanners in the scanning optical system 109 to be optically conjugated.

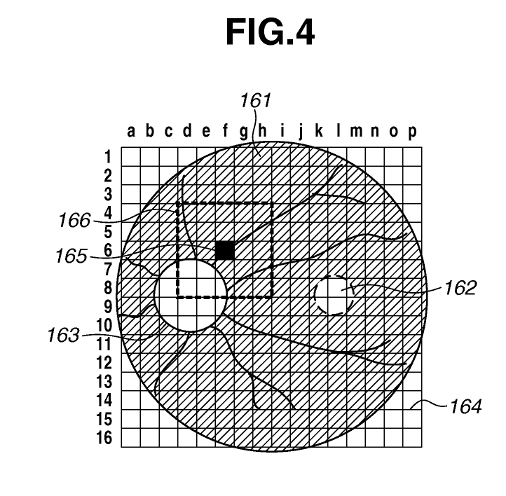

FIG. 4 is a diagram illustrating a state in which a fundus of the subject's eye is divided into a plurality of regions for the fundus imaging apparatus capturing images of the regions. A two-dimensional image illustrated in FIG. 4 includes a fundus 161, a macula 162, and an optic disk 163. A lattice 164 illustrates a state in which the fundus 161 is divided into a plurality of regions in a lattice pattern. Addresses a through p are allocated in a horizontal direction, whereas addresses 1 through 16 are allocated in a vertical direction. The fundus is divided into 256 regions (16×16=256), and the fundus imaging apparatus captures an image for each region. Moreover, the scanning optical system 109 reads the lattice pattern with each lattice region being divided into 256 pixels×256 pixels, each being a square having a length of 3 μm, in the main scanning direction and the sub-scanning direction, respectively. A user uses a mouse or a keyboard connected to a controller described below to designate an imaging region.

Referring back to FIG. 1, the measurement light 105 scanned by the scanning optical system 109 is emitted onto the eye 111 via eyepiece lenses 110-1 and 110-2. The measurement light emitted onto the eye 111 is reflected from or scattered by the fundus. A position of each of the eyepiece lenses 110-1 and 110-2 is adjusted. Such adjustments enable suitable light to be emitted according to a diopter of the eye 111. Herein, the lens is used for each of the eyepiece lenses 110-1 and 110-2. However, a spherical mirror may be used.

The fundus imaging apparatus further includes an optical spectrometer 119 serving as a beam splitter, and a fixation lamp 120. The beam splitter 119 guides the light from the fixation lamp 120 and the measurement light 105 to the subject's eye. The fixation lamp 120 guides a line of sight of the subject. The fixation lamp 120 includes, for example, a liquid crystal display 141, and light emitting diodes (LEDs) arranged in a lattice pattern on a plane.

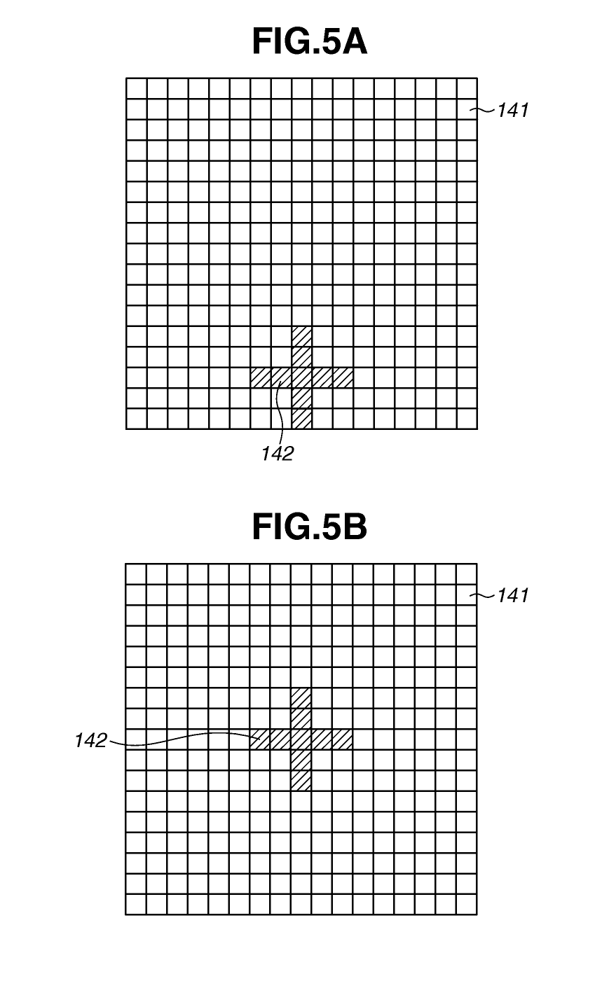

FIGS. 5A and 5B are enlarged views of the liquid crystal display 141 of the fixation lamp 120. As illustrated in FIG. 5A, a cross shape 142 is lit on the liquid crystal display 141. By causing the subject to gaze at an intersection of the cross shape 142, a movement of the subject's eye can be stopped. Moreover, by vertically and horizontally moving the lighting position of the cross shape 142 on the liquid crystal display 141, the line of sight of the subject can be controlled, so that a desired region of the subject's eye can be observed. On the liquid crystal display 141 of the fixation lamp 120, the cross shape 142 is lit in a target position corresponding to a region of the fundus that is to be captured. FIG. 5A illustrates a state in which the cross shape 142 is lit in a case where an upper center region of the fundus is to be captured.

In another example, the fixation lamp 120 displays the cross shape 142 at the center of the liquid crystal display 141 as illustrated in FIG. 5B. By changing the centers of rotation angles of the galvanometer scanner for the main scanning and the galvanometer scanner for the sub-scanning while the subject is continuously gazing at the front, an image of a region away from the center of the eye 111 can be captured.

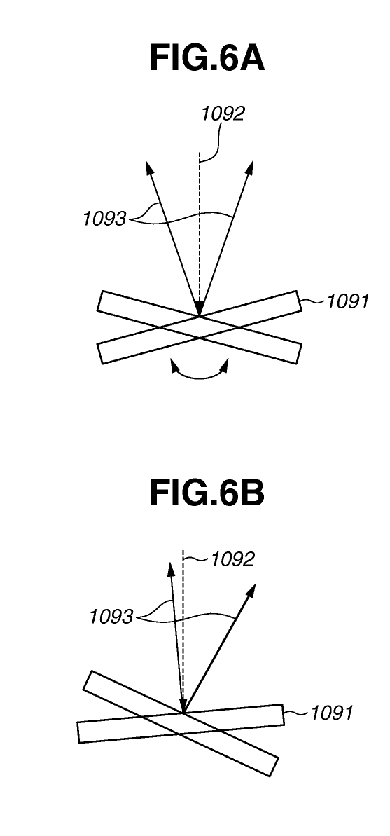

FIGS. 6A and 6B are diagrams illustrating a galvanometer scanner including a mirror 1091. As illustrated in FIGS. 6A and 6B, a change in a rotation range of the mirror 1091 of the galvanometer scanner changes a scanning angle of reflected light 1093 with respect to incident light 1092, thereby changing an imaging region of a fundus.

The light reflected from or scattered by a retina of the eye 111 travels along a path in an opposite direction to an incident direction. Then, the light is partially reflected from the wavefront sensor 115 by using the light splitting unit 106. The resultant light is used to measure a wavefront of a ray.

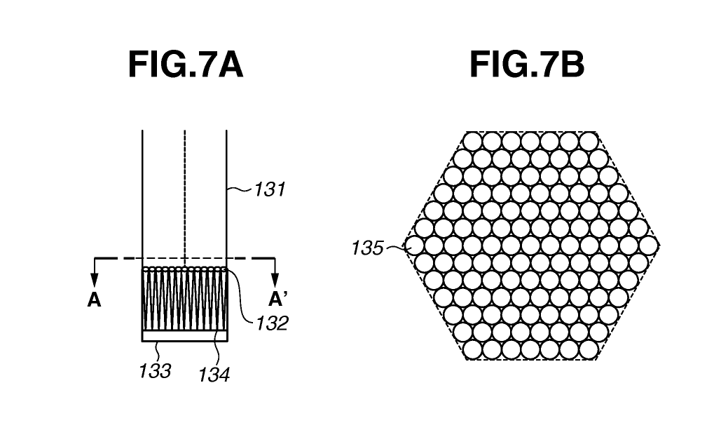

FIGS. 7A and 7B are schematic diagrams illustrating the wavefront sensor 115. In the present exemplary embodiment, a Shack-Hartman Sensor is used as the wavefront sensor 115. In FIG. 7A, a ray 131 is used to measure a wavefront. The ray 131 is condensed on a focal plane 134 on a CCD sensor 133 through a microlens array 132. FIG. 7B is a view as seen from a line A-A′ of FIG. 7A. In FIG. 7B, the microlens array 132 includes a plurality of microlenses 135. The ray 131 is condensed on the CCD sensor 133 via the respective microlenses 135. Thus, the ray 131 is divided and then condensed on spots being the same in number as the number of the microlenses 135.

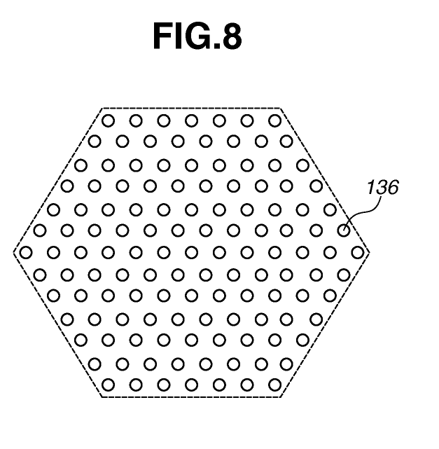

FIG. 8 is a diagram illustrating the CCD sensor 133. After passing through the microlenses 135, the rays are condensed on respective spots 136. Then, a wavefront of the ray entered from the position of each spot 136 is calculated.

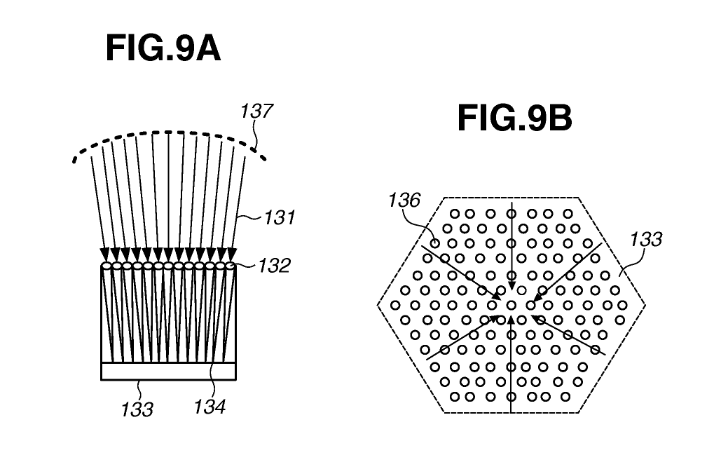

FIG. 9A is a schematic diagram illustrating a measurement result of a wavefront having a spherical aberration. The rays 131 form a wavefront as indicated by a dotted line 137. The rays 131 are condensed via the microlens array 132 on positions in a direction locally perpendicular to the wavefront. Herein, a light condensing state of the CCD sensor 133 is illustrated in FIG. 9B. Since the rays 131 have a spherical aberration, the rays 131 are condensed with the spots 136 arranged toward a center portion in a bias manner. Calculation of this position can detect the wavefront of the rays 131. In the present exemplary embodiment, the Shack-Hartman Sensor is used as the wavefront sensor 115. However, the present exemplary embodiment is not limited thereto. For example, another wavefront measurement unit such as a curvature sensor may be used. Alternatively, a method for determining a wavefront from a formed point image by inverse calculation may be used.

In FIG. 1, the reflected light transmitted through the light splitting unit 106 is partially reflected from the light splitting unit 104, and the resultant light is guided to a light intensity sensor 114 via a collimator 112 and an optical fiber 113. The light intensity sensor 114 converts the light into electric signals, and a control unit 117 forms an image as a fundus image and displays the resultant image on a display 118.

The wavefront sensor 115 is connected to an adaptive optics control unit 116. The wavefront sensor 115 notifies the adaptive optics control unit 116 of a received wavefront. The wavefront correcting device 108 is also connected to the adaptive optics control unit 116. The wavefront correcting device 108 performs modulation according to an instruction from the adaptive optics control unit 116. The adaptive optics control unit 116 calculates a modulation amount (a correction amount) such that the wavefront acquired by the wavefront sensor 115 is corrected to a wavefront having no aberration. Then, the adaptive optics control unit 116 instructs the wavefront correcting device 108 to perform modulation to correct the wavefront. The measurement of the wavefront and the instruction to the wavefront correcting device 108 are repeated to perform feedback control such that a suitable wavefront is constantly provided.

In the present exemplary embodiment, the adaptive optics control unit 116 models the measured wavefront into the Zernike function to calculate a coefficient for each order, and calculates a modulation amount of the wavefront correcting device 108 based on the coefficient. In the modulation amount calculation, based on a reference modulation amount for the wavefront correcting device 108 forming a shape of each Zernike order, the adaptive optics control unit 116 multiplies all the measured coefficients of Zernike order by the reference modulation amount. Moreover, the adaptive optics control unit 116 adds all the resultant values to determine a final modulation amount.

In the present exemplary embodiment, since the reflection-type liquid crystal spatial phase modulator having pixels of 600×600 is used as the wavefront correcting device 108, a modulation amount of each of 360,000 pixels is calculated according to the above calculation method. For example, if coefficients of a first order to a fourth order of the Zernike function are used for the calculation, the adaptive optics control unit 116 multiplies 14 coefficients by a reference modulation amount for 360,000 pixels. Herein, 14 coefficients are Z1−1, Z1+1, Z2−2, Z2−0, Z2+2, Z3−3, Z3−1, Z3+1, Z3+3, Z4−4, Z4−2, Z4−0, Z4+2, and Z4+4.

Moreover, if coefficients of a first order to a sixth order of the Zernike function are used for the calculation, the adaptive optics control unit 116 multiplies 27 coefficients by a reference modulation amount for 360,000 pixels. The 27 coefficients are Z1−1, Z1+1, Z2−2, Z2−0, Z2+2, Z3−3, Z3−1, Z3+1, Z3+3, Z4−4, Z4−2, Z4−0, Z4+2, Z4+4, Z5−5, Z5−3, Z5−1, Z5+1, Z5+3, Z5+5, Z6−6, Z6−4, Z6−2, Z6−0, Z6+2, Z6+4, and Z6+6.

Although most of the eye aberrations are low-order aberrations such as myopia, hyperopia, and astigmatism, there are high-order aberrations caused by minute unevenness of an eye's optical system or tear film irregularities. In a case where eye aberrations are expressed by Zernike function system including Zernike quadratic, cubic, quartic, quintic, and sextic functions, the Zernike quadratic function for myopia, hyperopia, and astigmatism is mostly used. The Zernike cubic and quartic functions are used in some cases, whereas higher functions such as quintic and sextic functions are barely used. Since part of the optical system includes a subject's eye, the optical system is in an uncertain state. Thus, a wavefront generally has difficulty in achieving a low aberration by one aberration measurement and one correction. The aberration measurement and correction are repeatedly performed until an aberration that allows imaging is acquired.

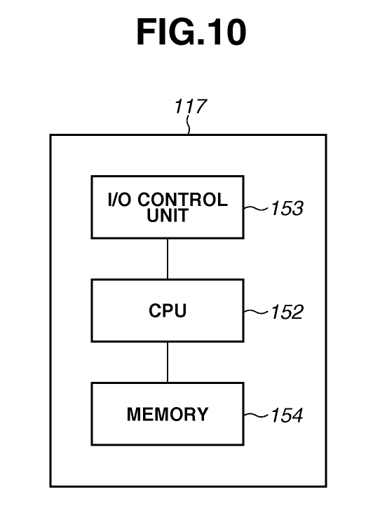

The fundus imaging apparatus is controlled by the control unit 117. FIG. 10 is a diagram illustrating the control unit 117. As illustrated in FIG. 10, the control unit 117 includes a central processing unit (CPU) 152, an input-output (I/O) control unit 153, and a memory 154. The CPU 152 controls the fundus imaging apparatus according to a program. The memory 154 stores aberration information of the subject's eye imaged by the fundus imaging apparatus for each imaging region of the subject's eye. More specifically, the memory 154 stores an address (f, 6) of an imaging region 165 illustrated in FIG. 4, and a correction value used when an image of the imaging region 165 is captured. The I/O control unit 153 drives, for example, a mouse (not illustrated), a keyboard (not illustrated), a bar code reader (not illustrated), the scanning optical system 109, the adaptive optics control unit 116, and the control unit 117 according to commands from the CPU 152. Moreover, the I/O control unit 153 controls communications.

The CPU 152 reads a program stored in the memory 154 to execute the program, whereby functions and processing of the fundus imaging apparatus are performed. The functions and the processing of the fundus imaging apparatus are described below.

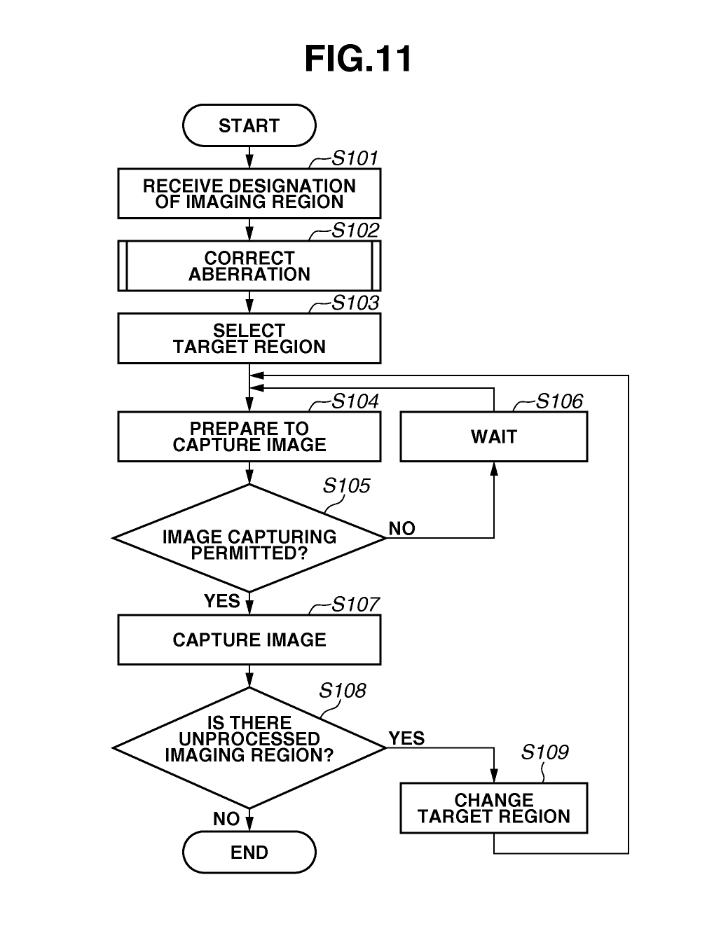

FIG. 11 is a flowchart illustrating imaging processing performed by the fundus imaging apparatus. An operator uses, for example, a mouse (not illustrated), a keyboard (not illustrated), and a bar code reader (not illustrated) to designate a plurality of imaging regions of a subject's eye, an imaging sequence of each imaging region, and the number of images to be repeatedly captured for each imaging region. In step S101, the CPU 152 receives the designation of the imaging regions of an imaging target, the imaging sequence, and the number of images to be captured.

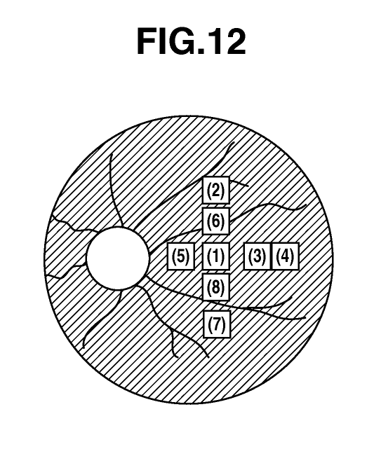

The fundus imaging apparatus continuously captures images of the same region until the number of imaging operations corresponding to the designated number of images to be captured is finished. The same region are imaged a plurality of times, and then the captured images are overlaid one another to form a clearer image. FIG. 12 is a diagram illustrating an imaging region of a subject's eye. In FIG. 12, eight imaging regions are designated, and numerical characters 1 through 8 indicate the imaging sequence of the respective imaging regions. When the fundus imaging apparatus starts an imaging operation, images of the first through eighth imaging regions are automatically captured in sequence.

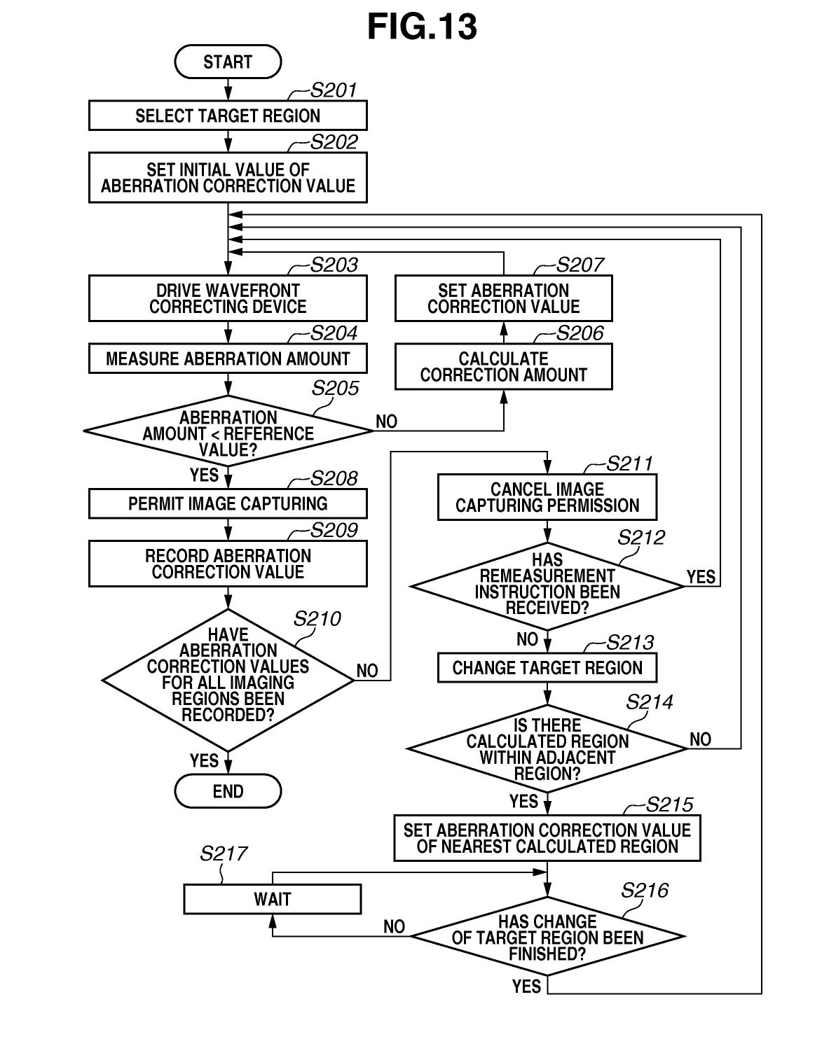

The description goes back to FIG. 11. After step S101, in step S102, the adaptive optics control unit 116 corrects an aberration. The aberration correction processing in step S102 will be described in detail with reference to a flowchart illustrated in FIG. 13. In step S201, the adaptive optics control unit 116 selects one imaging region from the plurality of imaging regions designated in step S101 (FIG. 11), as a target region of the aberration correction processing. In step S202, the adaptive optics control unit 116 performs modeling into a Zernike function to set a coefficient for each order, that is, an initial value, as an aberration correction value of the target region. Herein, the initial value is zero. However, in a case where there are unique aberrations in the wavefront correcting device 108 and other optical system members, the initial value may be a value for correcting such aberrations.

Subsequently, in step S203, the adaptive optics control unit 116 drives the wavefront correcting device 108 according to the aberration correction value set in step S201 to correct the aberration of the target region. In step S204, the adaptive optics control unit 116 measures an aberration amount using the wavefront sensor 115. In step S205, the adaptive optics control unit 116 determines whether the measured aberration amount is less than a reference value. Herein, the reference value is set beforehand in the memory 154, for example.

If the adaptive optics control unit 116 determines that the aberration amount is equal to or greater than the reference value (NO in step S205), the processing proceeds to step S206. In step S206, the adaptive optics control unit 116 calculates a modulation amount (a correction amount) such that the aberration amount is corrected. In step S207, the adaptive optics control unit 116 performs modeling into the Zernike function to calculate a coefficient for each order as an aberration correction value. The calculated aberration correction value is set in the adaptive optics control unit 116. The adaptive optics control unit 116 repeats the processing from steps S203 to S207 until the aberration amount becomes less than the reference value. If the adaptive optics control unit 116 determines that the aberration amount is less than the reference value (YES in step S205), the processing proceeds to step S208.

In step S208, the adaptive optics control unit 116 permits image capturing. In step S209, the adaptive optics control unit 116 records, onto the memory 154, position information indicating a position of the imaging region and the aberration correction value in association with a target region identification (ID).

Subsequently, in step S210, the adaptive optics control unit 116 determines whether the aberration correction values for all the designated imaging regions have been recorded. If the adaptive optics control unit 116 determines that the aberration correction values for all the imaging regions have already been recorded (YES in step S210), the aberration correction processing (S102) ends.

If the adaptive optics control unit 116 determines that there is an unprocessed imaging region (NO in step S210), the processing proceeds to step S211. In step S211, the adaptive optics control unit 116 cancels the image capturing permission. Then, in step S212, the adaptive optics control unit 116 determines whether an instruction for re-measurement of the aberration amount has been received from a user. If the adaptive optics control unit 116 determines that the re-measurement instruction has not been received (NO in step S212), the processing proceeds to step S213. If the adaptive optics control unit 116 determines that the re-measurement instruction has been received (YES in step S212), the processing returns to step S203. In such a case, the adaptive optics control unit 116 measures the aberration again to acquire a more appropriate aberration correction value.

In step S213, the adaptive optics control unit 116 changes the target region to an unprocessed imaging region. Herein, the adaptive optics control unit 116 identifies an imaging region the aberration correction value of which is not recorded in the memory 154, as the unprocessed imaging region. In step S214, the adaptive optics control unit 116 determines whether there is an imaging region with a calculated aberration correction value within an adjacent region of the new target region. Herein, the adjacent region represents a region that is defined based on a position of the target region as a reference. In the present exemplary embodiment, the adjacent region is defined as a region corresponding to 5×5 regions in a vertical direction and a horizontal direction around the target region. For example, as illustrated in FIG. 4, if the designated imaging region 165 has an address (f, 6), an adjacent region is defined as a region 166 surrounded by regions having addresses (d, 4), (h, 4), (d, 8), and (h, 8). The adjacent region is set beforehand in the memory 154, for example. Hereinafter, an imaging region the aberration correction value of which is already calculated is called “a calculated region”.

In a case where an aberration correction value associated with the address within the adjacent region has already be recorded in the memory 154, the adaptive optics control unit 116 determines that there is a calculated region. If the adaptive optics control unit 116 determines that there is a calculated region within the adjacent region (YES in step S214), the processing proceeds to step S215. If the adaptive optics control unit 116 determines that there is no calculated region within the adjacent region (NO in step S214), the processing returns to step S203.

In step S215, among the calculated regions within the adjacent region, the adaptive optics control unit 116 determines the calculated region that is positioned nearest to the imaging region to be processed, as a reference region. The adaptive optics control unit 116 determines an aberration correction value of the reference region as an initial value of an aberration correction value of the target region. The adaptive optics control unit 116 sets such an initial value of the aberration correction value as an aberration correction value of the target region. Herein, the processing in step S215 is one example of reference region determination processing, and one example of initial value determination processing. The reference region determination processing determines a reference region based on a distance between a calculated region and a target region, whereas the initial value determination processing determines an initial value of an aberration correction value of a target region.

Subsequently, in step S216, the adaptive optics control unit 116 determines whether a change of the imaging region has been finished. That is, the adaptive optics control unit 116 determines whether changes of the centers of rotation angles of the galvanometer scanner for the main scanning and the galvanometer scanner for the sub-scanning have been finished. If the adaptive optics control unit 116 determines that the change of the imaging region has not been finished (NO in step S216), the processing proceeds to step S217 in which the adaptive optics control unit 116 waits until the change of the imaging region is finished. If the adaptive optics control unit 116 determines that the change of the imaging region has been finished (YES in step S216), the processing returns to step S203.

In this manner, the adaptive optics control unit 116 does not correct an aberration until the change of the target region is finished. This is because, in a case where there is a significant difference between an aberration amount measured while a region is being moved and an aberration amount of an imaging region to be processed next, an aberration correction operation in the course of changing of the imaging region may cause the time needed for aberration correction to be longer.

As described above, the processing from steps S203 through S217 is repeated to complete the aberration corrections of all the designated imaging regions. The processing in step S215, and steps S203 through S207 subsequent to step S215 is one example of calculation processing performed by the adaptive optics control unit 116. With the calculation processing, the aberration correction value of the target region is calculated based on the aberration correction value of the calculated region.

The description goes back to FIG. 11. After the processing in step S102, in step S103, the CPU 152 selects a first imaging region designated in step S101, as an imaging region to be processed, i.e., as a target region. Herein, among the imaging regions designated in step S101, the first imaging region is the one designated to be imaged first according to the imaging sequence. In step S104, the CPU 152 prepares to capture an image of the target region. More specifically, as illustrated in FIG. 5B, the CPU 152 lights the cross shape 142 at the center of the liquid crystal display 141 of the fixation lamp 120. When the subject fixates the cross shape 142, the CPU 152 completes the preparation for imaging of the first region illustrated in FIG. 12.

According to the fundus imaging apparatus of the present exemplary embodiment, a position of the cross shape 142 to be lit is fixed to the center of the fixation lamp 120, as described above. The fundus imaging apparatus then changes the centers of rotations angles of the galvanometer scanner for the main scanning and the galvanometer scanner for the sub-scanning while the subject continuously fixates the front throughout the imaging period. Accordingly, the fundus imaging apparatus sequentially captures images of the designated imaging regions.

In step S105, the CPU 152 determines whether the adaptive optics control unit 116 has permitted the image capturing. If the CPU 152 determines that the image capturing has not been permitted (NO in step S105), the processing proceeds to step S106 in which the CPU 152 waits until the adaptive optics control unit 116 permits the image capturing. If the CPU 152 determines that the image capturing has been permitted (YES in step S105), the processing proceeds to step S107. In step S107, the CPU 152 controls the image capturing of the target regions. Through the process, fundus images of the target regions corresponding to the number of images that is designated in step S101 are obtained.

Subsequently, in step S108, the CPU 152 determines whether there is an unprocessed imaging region the image of which has not been captured. If the CPU 152 determines that there is an unprocessed imaging region (YES in step S108), the processing proceeds to step S109. If the CPU 152 determines that images of all the imaging regions have been captured (NO in step S108), the processing ends.

In step S109, the CPU 152 changes the target region to a next imaging region according to the imaging sequence. More specifically, the CPU 152 changes the centers of rotation angles of the galvanometer scanner for the main scanning and the galvanometer scanner for the sub-scanning to prepare for image capturing of the designated imaging region. Herein, as described above, the aberration correction operation is stopped in the course of changing of the imaging region. After the processing in step S109, the processing returns to step S104. In this manner, the processing from steps S104 through S109 is repeated, whereby images of all the designated imaging regions are captured.

For example, assume that first through eighth imaging regions illustrated in FIG. 12 are designated. In such a case, as for the second region, the fundus imaging apparatus starts an aberration correction using an aberration correction value of the first region as an initial value. As for the third region, the fundus imaging apparatus again starts an aberration correction using the aberration correction value of the first region as an initial value. As for the fourth region, the fundus imaging apparatus starts an aberration correction using the aberration correction value of the third region as an initial value.

As for the fifth region, the fundus imaging apparatus again starts an aberration correction using the aberration correction value of the first region as an initial value. As for the sixth region, the fundus imaging apparatus starts an aberration correction using the aberration correction value of the second region as an initial value. As for the seventh region, the fundus imaging apparatus again starts an aberration correction using the aberration correction value of the first region as an initial value. As for the eighth region, the fundus imaging apparatus starts an aberration correction using the aberration correction value of the seventh region as an initial value.

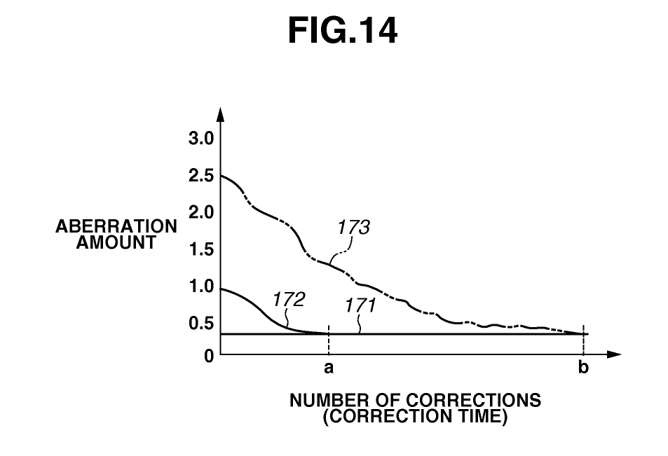

FIG. 14 illustrates a graph showing a relationship between the number of aberration corrections and an aberration amount. In FIG. 14, a horizontal axis indicates the number of aberration corrections, that is, the number of loops from steps S203 to S207 of the flowchart illustrated in FIG. 13. The number of aberration corrections represents the time necessary for correcting aberrations. A vertical axis indicates an aberration amount. A curved line 173 indicates a state of aberration corrections performed by a conventional method. A curved line 172 indicates a state of aberration corrections performed by the fundus imaging apparatus according to the present exemplary embodiment. A reference value 171 is used for comparing the size of the aberration amount in step S205 of the flowchart illustrated in FIG. 13. As illustrated in FIG. 14, in the conventional method, an imaging operation is started when a correction time b has elapsed since the beginning of correction processing. On the other hand, in the aberration correction according to the present exemplary embodiment, an imaging operation can be started at a correction time a.

Generally, in closed control of causing a control amount to converge on a target value, an increase in control gain can not only reduce a residual error, but also shorten the time needed for convergence. In particular, when control is started, a residual error with respect to a target value is large. Thus, an increase in the control gain in this period is effective for shortening the time needed for convergence. This corresponds to an abrupt change in the residual error. The abrupt change in the residual error indicates that such a change involves many high-frequency components.

In device responsiveness in general, high-frequency components respond with large delay. If such delay exceeds 180°, a residual error is increased although the residual error should be reduced. Consequently, the residual error cannot be converged. On the other hand, the fundus imaging apparatus according to the present exemplary embodiment can start convergence control when an initial residual error is small. Thus, the fundus imaging apparatus of the present exemplary embodiment is unlikely to be affected by delay occurring in a high-frequency component when the residual error changes, and oscillation is unlikely to occur even when control gain is increased. Moreover, the fundus imaging apparatus can shorten the time needed for convergence by increasing the control gain.

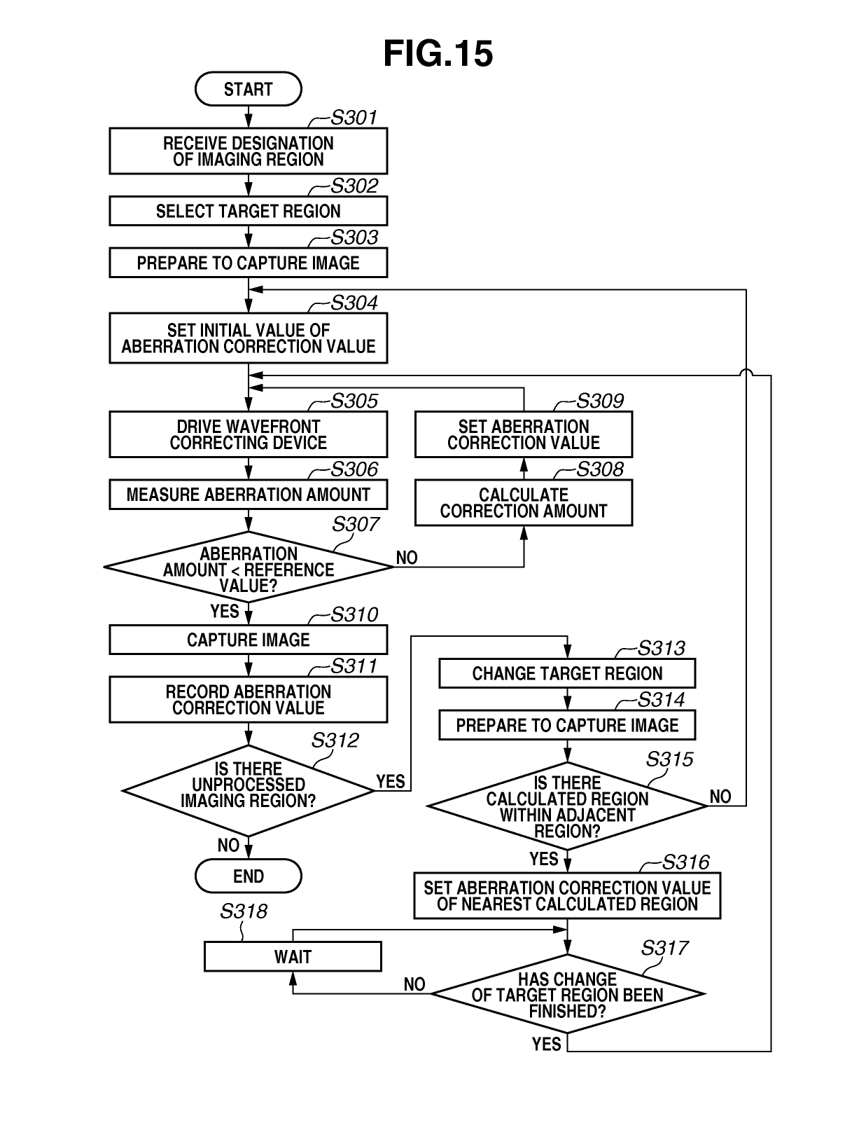

A fundus imaging apparatus according to a second exemplary embodiment successively perform an aberration correction and image capturing with respect to each imaging region. FIG. 15 is a flowchart illustrating imaging processing performed by the fundus imaging apparatus according to the second exemplary embodiment. In step S301, a CPU 152 receives designation of imaging regions, imaging sequence, and the number of images to be captured. In step S302, among the imaging regions designated in step S301, the CPU 152 selects a first imaging region as a target region. Subsequently, in step S303, the CPU 152 prepares to capture an image of the target region. In step S304, an adaptive optics control unit 116 sets an initial value as an aberration correction value of the target region.

The processing in steps S301, S302, S303, and S304 are similar to that in respective steps S101, S103, S104, and S202 described in the first exemplary embodiment.

In step S305, the adaptive optics control unit 116 drives a wavefront correcting device 108 according to the correction value set in step S304 to correct the aberration. In step S306, the adaptive optics control unit 116 measures an aberration amount using a wavefront sensor 115. Subsequently, in step S307, the adaptive optics control unit 116 determines whether the measured aberration amount is less than a reference value. Herein, the reference value is set beforehand in the memory 154, for example.

If the adaptive optics control unit 116 determines that the aberration amount is equal to or greater than the reference value (NO in step S307), the processing proceeds to step S308. In step S308, the adaptive optics control unit 116 calculates a modulation amount (a correction amount) such that the aberration amount is corrected. In step S309, the adaptive optics control unit 116 performs modeling into a Zernike function to calculate a coefficient for each order as an aberration correction value. The calculated aberration correction value is set in the adaptive optics control unit 116.

The processing from steps S305 to S309 of the present exemplary embodiment is similar to that from steps S203 through S207 described in the first exemplary embodiment, respectively. In the second exemplary embodiment, if the adaptive optics control unit 116 determines that the measured aberration amount is less than the reference value (YES in step S307), the processing proceeds to step S310.

In step S310, the CPU 152 controls the image capturing of the target regions. Through the process, fundus images of the target regions corresponding to the number of images that is designated in step S301 are obtained. Subsequently, in step S311, the adaptive optics control unit 116 records, onto the memory 154, position information indicating a position of the imaging region and the aberration correction value in association with a target region ID.

In step S312, the CPU 152 checks whether there is an unprocessed imaging region the image of which has not been captured. If the CPU 152 determines that there is an unprocessed imaging region (YES in step S312), the processing proceeds to step S313. If the CPU 152 determines that images of all the imaging regions have been captured (NO in step S312), the processing ends. In step S313, the CPU 152 changes the target region to a next imaging region according to the imaging sequence. In step S314, the CPU 152 prepares to capture an image of the changed target region. The processing in steps S310, S311, S313, and S314 according to the second exemplary embodiment is similar to that of respective steps S107, S209, S108, and S109 described in the first exemplary embodiment.

In step S315, the adaptive optics control unit 116 determines whether there is a calculated region within an adjacent region of the new target region. If the adaptive optics control unit 116 determines that there is a calculated region within the adjacent region (YES in step S315), the processing proceeds to step S316. If the adaptive optics control unit 116 determines that there is no calculated region within the adjacent region (NO in step S315), the processing returns step S304. In step S316, among the calculated regions within the adjacent region, the adaptive optics control unit 116 determines the calculated region that is positioned nearest to the imaging region to be processed (the calculate region at the shortest distance from the target region), as a reference region. The adaptive optics control unit 116 determines an aberration correction value of the reference region as an initial value of the aberration correction value of the target region, and sets such a value as the aberration correction value of the target region.

Subsequently, in step S317, the adaptive optics control unit 116 determines whether a change of the imaging region has been finished. If the adaptive optics control unit 116 determines that a change of the imaging region has not been finished (NO in step S317), the processing proceeds to step S318 in which the adaptive optics control unit 116 waits until the change of the target region is finished. If the adaptive optics control unit 116 determines that a change of the imaging region has been finished (YES in step S317), the processing returns to step S305. The processing from steps S315 through S318 of the present exemplary embodiment is similar to that from steps S214 through S217 described in the first exemplary embodiment, respectively.

As described above, the processing from steps S305 through S317 is repeated, whereby aberration corrections and image capturing for all the designated imaging regions can be successively performed. Other configurations and processing of the fundus imaging apparatus according to the second exemplary embodiment are similar to those of the fundus imaging apparatus according to the first exemplary embodiment.

A fundus imaging apparatus according to a third exemplary embodiment determines an initial value of an aberration correction value of a target region based on aberration correction values of a plurality of calculated regions and a distances between each of the plurality of calculated regions and the target region. More specifically, the fundus imaging apparatus applies a weight to an aberration correction value of each of the calculated regions according to the distance. Then, the fundus imaging apparatus calculates a sum of the aberration correction values of the respective calculated regions that are weighted according to the distance, and sets the resultant value as an initial value of an aberration correction value of a target region.

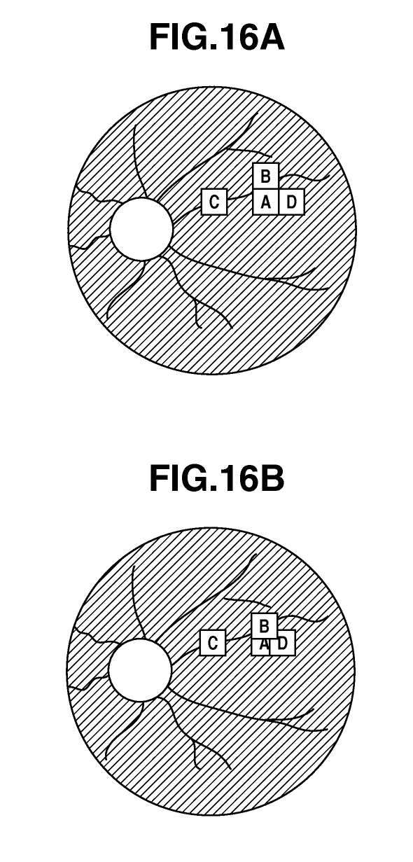

For example, as illustrated in FIG. 16A, a region A serves as a target region, whereas regions B, C and D serve as calculated regions within an adjacent region. Herein, assume that aberration correction values of the regions A, B, C, and D are a, b, c, and d, respectively, and distances from the region A to the regions B, C, and D are 2, 4, and 2, respectively. In such a case, the fundus imaging apparatus calculates the aberration correction value “a” of the region A by Equation 1.

a=b/2+c/4+d/2 (1)

Moreover, as illustrated in FIG. 16B, in a case where the regions B and D overlap the region A, the fundus imaging apparatus can calculate correction values according the respective distances from the region A.

Other configurations and processing of the fundus imaging apparatus according to the third exemplary embodiment are similar to those of the fundus imaging apparatuses of the other embodiments.

Moreover, in another exemplary case, if a distance between a calculated region that is acquired last among a plurality of calculated regions and a target region is equal to or greater than a threshold value, the fundus imaging apparatus may determine an initial value of an aberration correction value of the target region based on an aberration correction value of each of the plurality of calculated regions. Moreover, if the distance is less than the threshold value, the fundus imaging apparatus may determine an aberration correction value of the last calculated region as an initial value of an aberration correction value of the target region, and set such a value as the aberration correction value of the target region.

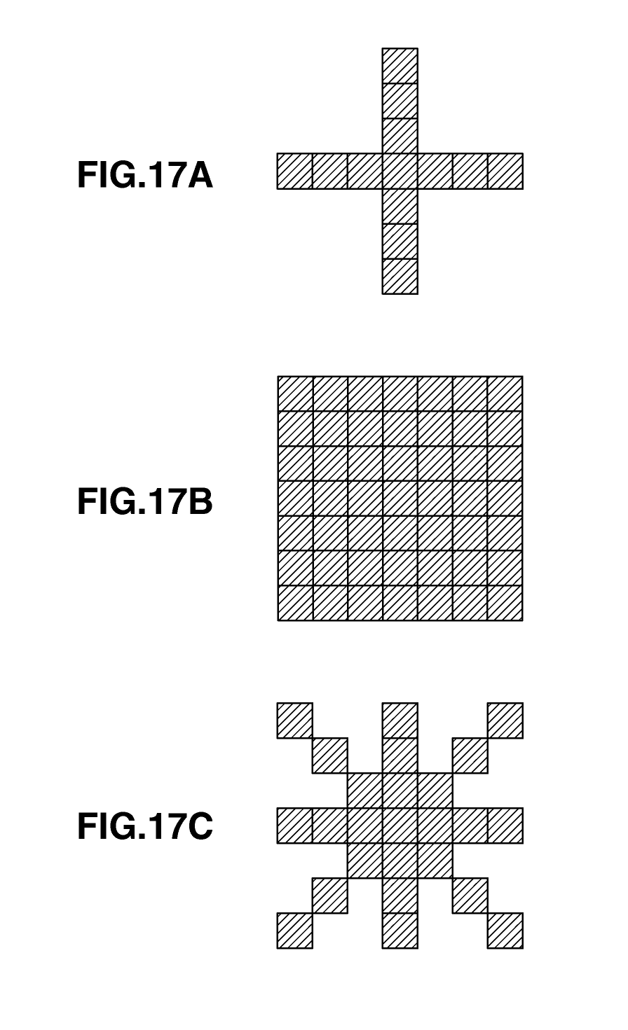

The fundus imaging apparatus according to the present exemplary embodiment individually receives designation of a plurality of imaging regions via a mouse, for example. However, this should not be construed in a limiting sense. Alternatively, as illustrated in FIGS. 17A, 17B, and 17C, the fundus imaging apparatus may automatically set imaging regions according imaging modes that are set beforehand. FIGS. 17A, 17B, and 17C are diagrams illustrating imaging regions that are set in different imaging modes. In this manner, in the fundus imaging apparatus, for example, a memory 154 stores setting information of the imaging region for each of the imaging modes illustrated in FIGS. 17A, 17B, and 17C.

In another exemplary case, a control unit 117 may function as an adaptive optics control unit 116. That is, the processing performed by the adaptive optics control unit 116 described in the present exemplary embodiment may be performed by a CPU 152 of the control unit 117.

Moreover, an exemplary embodiment of the present exemplary embodiment may be achieved by the processing below. That is, software (a program) for performing functions of each of the above present exemplary embodiments is supplied to a system or an apparatus via a network or various storage media. A computer (or a CPU and a micro processing unit (MPU)) of such a system or an apparatus reads the program to execute the processing.

According to each of the exemplary embodiments described above, the fundus imaging apparatus using an AO can shorten the time necessary for correcting an aberration of an eye.

Although exemplary embodiments have been described above, these exemplary embodiments are not seen to be limiting. The present disclosure encompasses all modifications and changes as described in the appended claims.

Exemplary embodiments can also be realized by a computer of a system or apparatus that reads out and executes computer executable instructions recorded on a storage medium (e.g., computer-readable storage medium) to perform the functions of one or more of the above-described embodiment(s), and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that these exemplary embodiments are not seen to be limiting.

This application claims the benefit of Japanese Patent Application No. 2014-142431, filed Jul. 10, 2014, which is hereby incorporated by reference herein in its entirety.

What is claimed is:

1. An fundus imaging apparatus comprising:
an imaging unit configured to capture images of a plurality of regions of a fundus of a subject's eye;
an initial value determination unit configured to determine an initial value of an aberration correction value of a target region based on an aberration correction value of at least one calculated region having a calculated aberration correction value from among the plurality of regions;
a measurement unit configured to measure an aberration of the target region based on the initial value;
a calculation unit configured to calculate an aberration correction value of the target region based on a measurement result of the measurement unit; and
an aberration correction unit configured to correct the aberration of the target region using the aberration correction value of the target region.

2. The fundus imaging apparatus according to claim 1, further comprising a reference region determination unit configured to, based on a distance between the at least one calculated region and the target region, determine a reference region to be referred to,
wherein the initial value determination unit determines the initial value of the aberration correction value of the target region based on the aberration correction value of the reference region.

3. The fundus imaging apparatus according to claim 2, wherein the reference region determination unit determines the at least one calculated region at a shortest distance from the target region as the reference region, and
wherein the initial value determination unit determines the aberration correction value of the reference region as the initial value of the aberration correction value of the target region.

4. The fundus imaging apparatus according to claim 2, wherein the initial value determination unit determines the initial value of the aberration correction value of the target region based on aberration correction values of respective reference regions and distances between the reference regions and the target region.

5. The fundus imaging apparatus according to claim 4, wherein the initial value determination unit determines a sum of the aberration correction values of the respective reference regions that are weighted according to the respective distances, as the initial value of the aberration correction value of the target region.

6. The fundus imaging apparatus according to claim 1, wherein the initial value determination unit determines the initial value of the aberration correction value of the target region based on the aberration correction value of the at least one calculated region and a distance between the at least one calculated region and the target region.

7. The fundus imaging apparatus according to claim 1, wherein the initial value determination unit determines the aberration correction value of the at least one calculated region that is imaged last as the initial value of the aberration correction value of the target region.

8. The fundus imaging apparatus according to claim 1, wherein the aberration correction unit does not correct the aberration during a period in which the target region is being changed.

9. A fundus imaging apparatus comprising:
an imaging unit configured to capture images of a plurality of regions of a fundus of a subject's eye;
an initial value determination unit configured to determine an aberration correction value of a first region having a calculated aberration correction value from among the plurality of regions as an initial value of an aberration correction value of a second region from among the plurality of regions;
a measurement unit configured to measure an aberration of the second region based on the initial value;
a calculation unit configured to calculate an aberration correction value of the second region based on a measurement result of the measurement unit; and
an aberration correction unit configured to correct the aberration of the second region using the aberration correction value of the second region.

10. The fundus imaging apparatus according to claim 9, wherein the measurement unit measures an aberration of the first region,
wherein the calculation unit calculates an aberration correction value of the first region based on a measurement result of the aberration of the first region by the measurement unit, and
wherein the initial value determination unit determines the aberration correction value of the first region calculated by the calculation unit as the initial value of the aberration correction value of the second region.

11. The fundus imaging apparatus according to claim 10, wherein, in a case where a distance between a third region and the second region from among the plurality of regions is less than a threshold value, the initial value determination unit determines the aberration correction value of the second region as an initial value of an aberration correction value of the third region,
wherein the measurement unit measures an aberration of the third region based on the initial value of the third region, and
wherein the calculation unit calculates an aberration correction value of the third region based on a measurement result of the aberration of the third region by the measurement unit.

12. The fundus imaging apparatus according to claim 10, wherein, in a case where a distance between a third region and the second region from among the plurality of regions is greater than or equal to a threshold value, the initial value determination unit determines an initial value of an aberration correction value of the third region based on the aberration correction value of the second region and the aberration correction value of the first region,
wherein the measurement unit measures an aberration of the third region based on the initial value of the third region, and
wherein the calculation unit calculates an aberration correction value of the third region based on a measurement result of the aberration of the third region by the measurement unit.

13. An aberration correction method executed by a fundus imaging apparatus, the aberration correction method comprising:
determining an initial value of an aberration correction value of a target region based on an aberration correction value of at least one calculated region having a calculated aberration correction value from among a plurality of regions of a fundus of a subject's eye;
measuring an aberration of the target region based on an initial value;
calculating an aberration correction value of the target region based on a measurement result of an aberration of the target region; and
correcting the aberration of the target region using the aberration correction value of the target region.

14. An aberration correction method executed by a fundus imaging apparatus, the aberration correction method comprising:
determining an aberration correction value of a first region having a calculated aberration correction value from among a plurality of regions of a fundus of a subject's eye as an initial value of an aberration correction value of a second region from among the plurality of regions;
measuring an aberration of the second region based on an initial value;
calculating an aberration correction value of the second region based on a measurement result of an aberration of the second region; and
correcting the aberration of the second region using the aberration correction value of the second region.

15. The aberration correction method according to claim 14, wherein an aberration correction value of the first region is calculated based on a measurement result of an aberration of the first region, and
wherein the aberration correction value of the first region calculated by the calculating is determined as the initial value of the aberration correction value of the second region.

16. The aberration correction method according to claim 15, wherein, in a case where a distance between a third region and the second region from among the plurality of regions is less than a threshold value, the aberration correction value of the second region is determined as an initial value of an aberration correction value of the third region, and
wherein an aberration correction value of the third region is calculated based on a measurement result of an aberration of the third region that is based on the initial value of the third region.

17. The aberration correction method according to claim 15, wherein, in a case where a distance between a third region and the second region from among the plurality of regions is greater than or equal to a threshold value, an initial value of an aberration correction value of the third region is determined based on the aberration correction value of the second region and the aberration correction value of the first region, and
wherein an aberration correction value of the third region is calculated based on a measurement result of an aberration of the third region that is based on the initial value of the third region.

18. A computer readable storage medium storing computer executable instructions for causing a computer to execute an aberration correction method, the aberration correction method comprising:
determining an initial value of an aberration correction value of a target region based on an aberration correction value of at least one calculated region having a calculated aberration correction value from among a plurality of regions of a fundus of a subject's eye;
measuring an aberration of the target region based on an initial value;
calculating an aberration correction value of the target region based on a measurement result of an aberration of the target region; and
correcting the aberration of the target region using the aberration correction value of the target region.

19. A computer readable storage medium storing computer executable instructions for causing a computer to execute an aberration correction method, the aberration correction method comprising:
determining an aberration correction value of a first region having a calculated aberration correction value from among a plurality of regions of a fundus of a subject's eye as an initial value of an aberration correction value of a second region from among the plurality of regions;
measuring an aberration of the second region based on an initial value;
calculating an aberration correction value of the second region based on a measurement result of an aberration of the second region; and
correcting the aberration of the second region using the aberration correction value of the second region.
